(19) United States
(12) Patent Application Publication
(10) Pub. No.: US 2005/0215516 A1
(43) Pub. Date: Sep. 29, 2005

(54) New free-radical scavenger containing viscoelastic composition, methods of use and package

(76) Inventors: Claudio Bucolo, Acicastello (Catania) (IT); Melina G. Cro, Catania (IT); Adriana L. A. Maltese, Tremestieri Etneo (Catania) (IT); Dharmendra M. Jani, Fairport, NY (US)

Correspondence Address: Bausch & Lomb Incorporated, One Bausch & Lomb Place, Rochester, NY, 14604-2701, US

(21) Appl. No.: 10/812,551

(22) Filed: Mar. 29, 2004

(51) Int. Cl. A61K 31/728; A61K 31/716

(52) U.S. Cl. 514/54; 514/57; 623/4.1

(57) ABSTRACT

The present invention is directed to a viscoelastic composition comprising an aqueous solution having a minimum of about 0.01% w/v and a maximum of about 20% w/v of a viscoelastic polymer based upon the total volume of the viscoelastic composition. Typically, the viscoelastic composition further contains tris[hydroxymethyl]aminomethane and a polyol (for example sorbitol). The present invention also includes methods of use of the new viscoelastic composition and a packaging device.

### BACKGROUND OF THE INVENTION

1. Field of the Invention

This invention relates to a viscoelastic composition, method of use and related device used in viscosurgical applications and more particularly to a viscoelastic composition used in ophthalmic surgical application such as cataract removal surgery.

2. Discussion of the Related Art

In the past decade, advances in the technology of eye surgery have made surgical treatment of eye disease and deformities attractive to alternative therapies. Cataract removal is one of the more common surgical procedures. Cataracts are opacities of the ocular lens, which generally arise in the elderly. Typically, cataract surgery involves removal of the cataractous lens from the capsular bag and replacement of the cataractous lens with a synthetic intraocular lens. Presently, this procedure involves making an incision through the sclera into the anterior chamber of the patient's eye. Another incision is made into the capsular bag. The cataractous lens is fractured in the capsular bag by procedures such as phacoemulsification and removed from the capsular bag by procedures such as aspiration. Thereafter an intraocular lens is inserted into the capsular bag and deployed therein.

The overall procedure is potentially traumatic to the capsular bag and the tissue surrounding the anterior chamber. It is advantageous to reduce the amount of trauma to any living tissue in the patient's eye during a surgical procedure. Particularly, lens endothelial cells in the capsular bag are sensitive to damage. Damage to the lens endothelial cells is often permanent. Serious damage can cause loss of eyesight and failure of the surgical procedure.

Moreover, the process of phacoemulsification produces free radicals and/or oxidants. Free radicals and/or oxidants are unstable and react somewhat indiscriminately with biological molecules in tissue. For example, a free radical and/or oxidant that are produced in phacoemulsification can damage proteins, cell walls or even the DNA of a cell. It is advantageous to reduce the damage caused by these free radicals and/or highly reactive ions.

Viscoelastic compositions are injected in the anterior chamber of the eye and the capsular bag during surgery to protect the tissue from physical trauma. The viscoelastic compositions provide a physical barrier or cushion between the instruments and the tissue. Furthermore, viscoelastic compositions maintain the shape of a cavity during operation including the anterior chamber and capsular bag. Viscoelastic compositions have been known to contain agents that are free radical scavengers and/or antioxidants.

Selection of an ingredient in a viscoelastic composition for the purpose of controlling free-radical activity and/or antioxidants, require satisfying several criteria. The ingredient cannot negatively impact the viscoelastic properties, irritate tissue or cause an adverse immune response. The ingredient should be effective as a free-radical scavenger and/or antioxidant under conditions of desirable pH and osmolality. Of course, the effectiveness of the free-radical scavenger to dampen free radical activity is an important factor.

U.S Pat. No. 5,880,107 discloses a viscoelastic composition for use in eye surgery. The viscoelastic composition contains hyaluronic acid as the primary ingredient to provide appropriate viscoelasticity. The composition further contained a citric acid salt, typically tri-sodium citrate, an antioxidant tolerated by the intraocular tissues and a phosphate buffer. The antioxidant is selected from the group comprising glucose, sulphides, superoxide dismutase (SOD), cysteine and derivates thereof. Furthermore, other antioxidants that could be used include antioxidants, which have at least one —SH or —CHO group, peptides and enzymes.

U.S. Pat. No. 6,086,597 discloses a sodium hyaluronate viscosurgical composition that contains a compound to as a scavenger including superoxidedismutase, mannitol and glutathione.

U.S. Pat. No. 5,631,243 discloses a collagen-based viscosurgical composition. The composition has higher solubility at pH values close to neutral pH. Osmolarity is increased using nonionic solutes including glycerol, sorbitol, xylitol, threitol, mannitol, etc.

Tris[hydroxymethyl]-aminomethane is a quaternary ammonium compound that is found as an ingredient in a buffer system in topical ophthalmic formulations. See U.S. Publ. No. 2003-0232089 and WO03/072081.

While significant improvements have been made in the Theological properties of viscoelastic compositions, there still exists a need for a composition that reduces the free radical and/or oxidant quenching activity without negatively impacting the viscoelastic properties of the viscoelastic composition. The present invention addresses these and other needs.

### SUMMARY OF THE INVENTION

The present invention is directed to a viscoelastic composition comprising an aqueous solution having a minimum of about 0.01% w/v and a maximum of about 20% w/v of a viscoelastic polymer based upon the total volume of the viscoelastic composition. Typically, the viscoelastic composition further contains tris[hydroxymethyl]aminomethane. Preferably, the viscoelastic composition and viscoelastic polymer is viscosurgically pure.

In one embodiment, the viscoelastic composition further comprises a polyol, including but not limited to pentahydric alcohols, hexahydric alcohols and heptahydric alcohols and mixtures thereof. In one embodiment, the polyol is mannitol or sorbitol or mixtures thereof.

In one embodiment, there is a method of maintaining space in a cavity in human tissue. The method comprises the step of injecting, into the cavity, a viscoelastic composition according to any embodiment, aspect, feature, combination or concept disclosed herein. Thereafter, the viscoelastic composition is removed from the cavity. Preferably, the cavity is the anterior chamber of the eye or the capsular bag.

In still another embodiment, there is a method of protecting tissue from trauma during a surgical procedure. The method comprises the step of coating at least a portion of the tissue with a viscoelastic composition according to any embodiment, aspect, feature, combination or concept disclosed herein. A surgical procedure is then performed near the tissue. When the surgical procedure is completed, at least a portion of the viscoelastic composition is removed from the tissue.

In one embodiment, there is a method of replacing a natural lens from an eye. The method comprises providing a passage through a sclera into an anterior chamber of the eye. At least a portion of the aqueous humor is removed from the anterior chamber. A viscoelastic composition according to any embodiment, aspect, feature, combination or concept disclosed in this application is injected into the anterior chamber. The lens in the capsular bag of the eye is removed by, for example, phacoemulsification. Substantially all of the lens is removed from the capsular bag. The viscoelastic composition is injected into the capsular bag. An intraocular lens is inserted into the capsular bag. Thereafter, at least a portion of the viscoelastic composition is removed from the capsular bag and/or the anterior chamber—typically by aspiration. The sclera is then sutured or closed after the viscoelastic composition is removed, at least in part, from the anterior chamber.

In another embodiment, there is a package for a viscoelastic composition, the package comprising a syringe containing a viscoelastic composition according to any embodiment, aspect, feature, combination or concept disclosed herein.

### DETAILED DESCRIPTION OF THE INVENTION

Introduction

The present invention is directed to a viscoelastic composition comprising an aqueous solution having a minimum of about 0.01% w/v and a maximum of about 20% w/v of a viscoelastic polymer based upon the total volume of the viscoelastic composition. Typically the viscoelastic composition further contains tris[hydroxymethyl]aminomethane. The present invention also includes methods of use and a device.

Definitions

Viscosurgically pure as it pertains to a viscoelastic composition or ingredient thereof is defined as a level of purity that is sufficiently free of impurities to meet or exceed the United States Food and Drug Administration standards for a viscosurgical viscoelastic effective at the time this application is filed.

Polysaccharides are defined as saccharides that have 10 or more saccharide monomer units.

Zero-shear viscosity is defined as the extrapolation of the viscosity of a liquid to a zero-shear rate from measurements of viscosity as the shear rate approaches zero measured on a plate and cone rheometer at 34° C.

High-shear viscosity is defined as the viscosity of a liquid measured on a plate and cone rheometer at 34° C. with a shear rate of 300 s⁻¹.

Pseudoplastic material is defined as a material that has relatively high viscosity under low-shear and relatively low viscosity under high-shear conditions.

The phrase “removing substantially all”, as it relates to lenses and lens fragments, is defined as removing a sufficient quantity that an effective implantation of an intraocular lens is not inhibited thereafter. According to one embodiment, an effective removal of the lens requires a minimum of 90% w/v of the lens, 95% w/v of the lens or 98% w/v of the lens.

A cannula is defined as any tubular member having a passage that is configured to penetrate tissue and deliver a device through the passage.

A polyol for the purpose of this application is defined as a hydrocarbon having a hydroxyl group attached to each of the carbon atoms of the hydrocarbon.

A pentahydric alcohol is defined as a linear polyol having five carbon atoms.

A hexahydric alcohol is defined as a linear polyol having six carbon atoms.

A heptahydric alcohol is defined as a linear polyol having seven carbon atoms.

The percentage of quenching as describe in the application with the exception of the examples is defined as the percentage amount that free-radical activity is prevented as evaluated by the 2-deoxy-D-ribose (2-DR) oxidation method. This is a conventional method of OH-radical detection forming by the Fenton reaction, radiation or ultrasound. It is based on its reaction with 2-DR. The obtained product of degradation, after a thermoactivated reaction with thiobarbituric acid (TBA), produces a pink chromogen quantified by HPLC.

Formulation

According to one embodiment of the present invention, there is a viscoelastic composition comprising an aqueous solution having a minimum of about 0.01% w/v and a maximum of about 20% w/v of a viscoelastic polymer based upon the total volume of the viscoelastic composition. Typically the viscoelastic composition further contains tris[hydroxymethyl]aminomethane.

In one embodiment, the viscoelastic composition has a concentration of tris[hydroxymethyl]aminomethane that is a maximum of about 50 mM and a minimum of about 0.1 mM based upon the total weight of the viscoelastic composition. Typically, the concentration of tris[hydroxymethyl]aminomethane is a maximum of about 30 mM and a minimum of about 0.5 mM based upon the total volume of the viscoelastic composition. Preferably, the concentration of tris[hydroxymethyl]aminomethane is a minimum of about 0.5 mM, about 0.7 mM or about 0.9 mM and a maximum of about 15 mM, about 20 mM or about 25 mM based upon the volume of the viscoelastic composition in one aspect of the invention.

In one embodiment, the viscoelastic composition also contains a polyol. The polyol in the viscoelastic composition, optionally, is selected from the group comprising pentahydric alcohols, hexahydric alcohols and heptahydric alcohols and mixtures thereof. Preferably, the polyol is a hexahydric alcohol. More preferably, the polyol is mannitol and/or sorbitol.

The viscoelastic composition has a concentration of the polyol, including but not limited to pentahydric alcohols, hexahydric alcohols and heptahydric alcohols and mixtures thereof, that is a minimum of about 0.1% w/v and a maximum of about 15% w/v based upon the total volume of the viscoelastic composition. Typically, the concentration of the polyol including but not limited to pentahydric alcohols, hexahydric alcohols and heptahydric alcohols and mixtures thereof, is a minimum of about 0.3% w/v, about 0.5% w/v or about 1% w/v and a maximum of about 10% w/v, about 6% w/v or about 4% w/v based upon the total volume of the viscoelastic composition. Optionally, the concentration of mannitol and or sorbitol is at one or more concentrations in the range disclosed above for polyols.

The viscoelastic composition of one embodiment of the present invention has a ratio of the viscosity of the viscoelastic composition to the viscosity of a comparable viscoelastic composition having no polyol and tris[hydroxymethyl]aminomethane that is a minimum of about 1 and a maximum of about 2.5. A comparable viscoelastic composition is defined as a viscoelastic composition that has all of the same chemical ingredients as the viscoelastic composition at the same concentrations except it has no polyol and tris[hydroxymethyl]aminomethane. Typically, the ratio of the viscosity of the viscoelastic composition to the viscosity of a comparable viscoelastic composition is a minimum of about 1, about 1.1 and about 1.2 and a maximum of about 2.5, about 2.2 and about 2.

The viscoelastic composition of yet another embodiment quenches chemical scavengers effectively, wherein the percentage of quenching is a minimum of about 75%. Typically, the percentage quenching is greater than about 80% , about 85% , about 87% , about 90% or about 92% according to the method of testing in Example 9 herein.

The viscoelastic composition comprises one or more viscoelastic polymers that are useful and known as viscosurgical devices. In one embodiment, the viscoelastic polymer is selected from the group comprising hyaluronic acid, hydroxypropylmethylcellulose, polyacrilyc acid, carbopol, polyvinylalchol, polyvinylpirrolidone, condroitin sulfate, polycarbophil, methylcellulose, carboxymethylcellulose, hydroxyethylcellulose, hydroxypropylcellulose, ethylcellulose, polyethylene oxides, alginate, pectin, xanthan gum, dextrans, collagen and derivatives threof and salts thereof and combinations thereof.

In one embodiment, the average molecular weight of the viscoelastic polymer, including a polysaccharide, is a minimum of about 20 kD and a maximum of about 5,000 kD. Generally, the average molecular weight of a viscoelastic polymer, including polysaccharide, is a minimum of about 30 kD, about 50 kD, about 70 kD, about 400 kD, about 500 kD, about 750 kD or about 1,000 kD. Typically, the average molecular weight of a viscoelastic polymer, including a polysaccharide, is a maximum of about 50 kD, about 80 kD, about 100 kD, about 200 kD, about 400 kD, about 500 kD, about 1,000 kD or about 3,000 kD.

Typically, there are two general classes of viscoelastic compositions. A dispersive viscoelastic composition has properties that disperse or coat the tissue well and adhere well to the tissue. A dispersive viscoelastic composition (also known as an “adhesive viscoelastic composition”) typically has a low molecular weight. A cohesive viscoelastic composition is better at maintaining the space in a cavity in human tissue and is less likely to leak from the cavity under low or zero shear conditions. Typically, a cohesive viscoelastic composition has a high molecular weight.

In one embodiment, the average molecular weight of a viscoelastic polymer in a dispersive viscoelastic composition is a minimum of about 20 kD, 30 kD, about 50 kD or about 70 kD. Typically, the average molecular weight of a viscoelastic polymer in a dispersive viscoelastic composition is a maximum of about 50 kD, about 80 kD, about 100 kD, about 200 kD, about 400 kD or about 500 kD.

In another embodiment, the average molecular weight of a viscoelastic polymer in a cohesive viscoelastic composition is a minimum of about 400 kD, about 500 kD, about 750 kD or about 1,000 kD. Typically, the average molecular weight of a viscoelastic polymer in a cohesive viscoelastic composition is a maximum of about 1,000 kD, 3,000 kD or about 5,000 kD.

The concentration of the viscoelastic polymer is a minimum amount of about 0.01% w/v and a maximum amount of about 20% w/v based upon the total weight of the viscoelastic composition in one embodiment. Typically, the concentration of the viscoelastic polymer is a minimum of about 0.1% w/v, about 0.2% w/v, about 1.0 or about 2.0% w/v and a maximum of about 0.3% w/v, about 0.5% w/v, about 1% w/v, about 2% w/v about 3% w/v, about 5% w/v or about 15% w/v based upon the total weight of the viscoelastic composition.

In still another embodiment, the viscoelastic polymer comprises a mixture of hyaluronic acid and/or salts thereof and hydroxypropylmethylcellulose.

The concentration of hyaluronic acid and/or salts thereof is a minimum of about 0.1% w/v and a maximum of about 6% w/v based upon the volume of the viscoelastic composition in one embodiment. Typically, the concentration of hyaluronic acid and/or salts thereof is a minimum of about 0.3% w/v, about 0.6% w/v or about 1% w/v and a maximum of about 6% w/v, about 4% w/v or about 2% w/v based upon the volume of the viscoelastic composition.

The average molecular weight of the hyaluronic acid and/or salts thereof is a minimum of about 500 kD and a maximum of about 5000 kD in one embodiment. Typically, the average molecular weight of the hyaluronic acid and/or salts thereof is a minimum of about 500 kD, about 700 kD or about 1000 kD and a maximum of about 4000 kD, about 300 kD or about 2000 kD.

The concentration of hydroxypropylmethylcellulose is a minimum of about 0.05% w/v and a maximum of about 5% w/v based upon the volume of the viscoelastic composition in one embodiment. Typically, the concentration of hydroxypropylmethylcellulose is a minimum of about 0.2% w/v, about 0.4% w/v or about 0.8% w/v and a maximum of about 5% w/v, about 3% w/v or about 1% w/v based upon the volume of the viscoelastic composition.

The average molecular weight of the hydroxypropylmethylcellulose is a minimum of about 10 kD and a maximum of about 120 kD according to one embodiment. Typically, the average molecular weight of the hydroxypropylmethylcellulose is minimum of about 10 kD, about 12 kD or about 20 kD and a maximum of about 120 kD, about 90 kD or about 86 kD.

In one embodiment, the viscoelastic polymer comprises a polysaccharide. In another embodiment, the viscoelastic polymer is preferably a polysaccharide selected from the group comprising hyaluronic acid, hydroxypropylmethylcellulose, condroitin sulfate, methylcellulose, carboxymethylcellulose, hydroxyethylcellulose, hydroxypropylcellulose, ethylcellulose, alginate, pectin, dextrans, collagen, proteoglycans, polyvinylpyrrolidone, keratin carrageenans and derivatives thereof and salts thereof and combinations thereof.

The viscoelastic polymer comprises alginate in one embodiment. Typically the concentration of alginate is a minimum of about 0.05% w/v and a maximum of about 9% w/v based upon the volume of the viscoelastic composition. Optionally, the minimum alginate concentration is about 1% w/v, about 1.5% w/v, about 2% w/v, about 3% w/v or about 4% w/v based upon the total weight of the viscoelastic composition. Optionally, the maximum alginate concentration is about 10% w/v, about 8% w/v, about 6% w/v, about 4% w/v, about 3% w/v or about 2% w/v based upon the total weight of the viscoelastic composition. Preferably, the alginate concentration is a minimum of about 2% w/v and a maximum of about 5.25% w/v.

In one embodiment, the average molecular weight of the alginate is a minimum of about 50 kD and a maximum of about 5,000 kD. Typically, the average molecular weight of the alginate is a minimum of about 100 kD, about 200 kD, about 500 kD or about 1000 kD. Typically, the average molecular weight of the alginate is a maximum of about 2000 kD, about 1000 kD, about 750 kD or about 500 kD.

The viscoelastic composition has one or more properties including but not limited to osmolality, pH, zero-shear viscosity and high-shear viscosity. The osmolality of the viscoelastic composition is a minimum of about 200 mOsmol/Kg and a maximum of about 400 mOsmol/Kg in an embodiment. Typically, the osmolality of the viscoelastic composition is a minimum of about 220 mOsmol/Kg, about 260 mOsmol/Kg, about 280 mOsmol/Kg, about 300 mOsmol/Kg or about 320 mOsmol/Kg and a maximum of about 400 mOsmol/Kg, about 380 mOsmol/Kg, about 360 mOsmol/Kg or about 340 mOsmol/Kg.

The zero-shear viscosity of the viscoelastic composition is a minimum of about 6·10⁴ cps and a maximum of about 4·10⁶ cps. Generally, the zero-shear viscosity of the viscoelastic composition is a minimum of about 6×10⁴ cps, about 4×10⁵ cps or about 8×10⁵ cps and a maximum of about 3.5×10⁶ cps, about 1.8×10⁶ cps or about 1.2×10⁶ cps.

The high-shear viscosity of the viscoelastic composition is a minimum of about 500 cps and a maximum of about 2000 cps. Generally, the high-shear viscosity of the viscoelastic composition is a minimum of about 500 cps, about 600 cps or about 700 cps and a maximum of about 2000 cps, about 1500 cps or about 1000 cps.

The pH of the viscoelastic composition of one embodiment is a minimum of about 5 and a maximum of about 8. In one embodiment, the pH of the viscoelastic composition is a minimum of about 5.5, about 6 or about 6.5 and a maximum of about 7.5, about 7.2 or about 7.

The viscoelastic composition of one embodiment has a formulation set forth in Table 1.

TABLE 1

| Component or Property of the | |
|---|---|
| Viscoelastic Composition | Amount |
| 1.0 × 10⁶-3 × 10⁶ Molecular Weight | 0.5% w/v to 3% w/v |
| Hyaluronic Acid or Salt Form Thereof | |
| 20,000-200,000 Molecular Weight Hydroxy- | 0.1% w/v to 2% w/v |
| propylmethylcellulose | |
| Sorbitol | 0.1% w/v to 20% w/v |
| Tris[hydroxymethyl]aminomethane | 1 mM to 100 mM |
| Buffered to pH | 6.9 to 7.5 |
| Osmolality adjusted to | 290-350 mOsm/Kg |

In one preferred embodiment the viscoelastic composition comprises the following:
2.3% w/v hyaluronic acid (MW 1.98×10⁶)
0.8% w/v hydroxypropylmethylcellulose (MW 86,000)
4.4% w/v sorbitol
20 mM tris[hydroxymethyl]aminomethane
purified water q. s. to 100 ml
pH 7.3
335 mOsm/Kg

In another preferred embodiment, the viscoelastic composition comprises the following:
2% w/v hyaluronic acid (MW 1.98×10⁶)
0.8% w/v hydroxypropylmethylcellulose (MW 86,000)
4.4% w/v sorbitol
20 mM tris[hydroxymethyl]aminomethane
purified water q. s. to 100 ml
pH 7.3
335 mOsm/Kg

In another preferred embodiment, the viscoelastic composition comprises the following:
2% w/v hyaluronic acid (MW 1.98×10⁶)
1% w/v hydroxypropylmethylcellulose (MW 86,000)
4.4% w/v sorbitol
20 mM tris[hydroxymethyl]aminomethane
purified water q. s. to 100 ml
pH 7.3
335 mOsm/Kg

In another preferred embodiment, the viscoelastic composition comprises the following:
5.25% w/v alginate
4.4% w/v sorbitol
20 mM tris[hydroxymethyl]aminomethane
Purified water q. s. a. d. to 100% w/v
   Methods of Use

Viscoelastic composition according to any one or more of the foregoing embodiments, concepts or aspects including combinations and variations of the foregoing embodiments can be used according to the following method or methods.

In one embodiment, there is a method of maintaining space in a cavity in human tissue. The method comprises the step of injecting, into the cavity, a viscoelastic composition according to any embodiment, aspect, feature, combination or concept disclosed herein. Thereafter, the viscoelastic composition is removed from the cavity. Preferably, the cavity is the anterior chamber of the eye or the capsular bag.

In still another embodiment, there is a method of protecting tissue from trauma during a surgical procedure. The method comprises the step of coating at least a portion of the tissue with a viscoelastic composition according to any embodiment, aspect, feature, combination or concept disclosed herein. Preferably, the tissue that is covered is in the anterior chamber of the eye and/or the capsular bag. A surgical procedure is then performed near the tissue. When the surgical procedure is completed, at least a portion of the viscoelastic composition is removed from the tissue.

In one embodiment, there is a method of replacing a natural lens from an eye. Examples of procedures for removing a lens from a patient's eye include but are not limited to U.S. Pat. No. 3,589,363 (cataract surgery), U.S. Pat. No. 3,693,613 (phacoemulsification) and U.S. Pat. No. 5,718,676 (process using micro flow needle), which are all incorporated herein by reference in their entirety. The process generally includes providing a passage through a sclera or cornea into an anterior chamber of the eye. The process involves making a small incision into the sclera or cornea. Alternatively or additionally, a cannula or trochar is used to create a passage through the sclera or cornea. Preferably, the incision or passage is as small as possible. Preferably, the incision or passage is smaller than about 5 mm, about 4 mm or about 3mm. Thereafter, the aqueous humor is withdrawn or otherwise removed from the anterior chamber of the eye.

A viscoelastic composition according to any one of the embodiments, aspects concepts, combinations or features is inserted into the anterior chamber. The viscoelastic composition maintains the space in the anterior chamber. The viscoelastic composition coats the tissue in the wall of the anterior chamber.

According to one embodiment, there is a package for a viscoelastic composition that includes a delivery device. The device delivers a viscoelastic composition into the anterior chamber of a patient's eye. The device includes a syringe that contains a viscoelastic composition according to any embodiment, aspect, combination, concept or feature disclosed herein.

The syringe further comprises an outlet port and, optionally, a cannula configured to sealably connect to the outlet port. The cannula has a maximum inner diameter of about 2 mm. Typically, the maximum inner diameter is about 1.8 mm, about 1.5 mm or about 1 mm. Generally, the minimum inner diameter is about 0.8 mm, about 0.6 mm or about 0.4 mm.

In one embodiment, the viscoelastic composition requires a maximum force of 30 N to pass through a stainless steel cannula having a length of 2.2 cm and an inner diameter of 0.5 mm at a delivery rate of 0.02 ml/sec. Preferably, the viscoelasfic composition requires a maximum force of about 27 N, about 25 N, about 20 N or about 18 N to pass through a stainless steel cannula having a length of 2.2 cm and an inner diameter of 0.5 mm at a delivery rate of 0.02 ml/sec.

Once the viscoelastic composition is inserted into the anterior chamber the corneal lens is removed. The technique for removing the lens includes performing a capsulorhexis incision and breaking down the lens into smaller pieces through phacoemulsification or other known techniques. Thereafter, the pieces are removed by, for example, aspiration.

The viscoelastic composition is inserted into the capsular bag for space maintenance purposes. Moreover, the viscoelastic composition coats the capsular bag and protects it for additional steps in the surgical procedure.

According to one embodiment, the intraocular lens is inserted into the capsular bag. Typically, there is a method of inserting an intraocular lens into a capsular bag of an eye. The method comprises providing a lens insertion device comprising a loadable chamber configured to receive the intraocular lens, a tapered conduit having a first end connected to the loadable chamber and a second end. The second end is configured to penetrate through the passage in the corneal lens and into the capsular bag. An example of a lens insertion device is found in U.S. Pat. No. 6,558,419, which is incorporated herein by reference in its entirety. The lens insertion device is further configured with a slidable actuator. The slidable actuator of one embodiment is configured to actuate the intraocular lens through the conduit past the second end. Typically, the second end of the tapered conduit has an inner diameter that is a maximum of about 5 mm. Preferably the second end of the tapered conduit has an inner diameter that is a maximum of about 4 mm about 3.5 mm, about 3 mm or about 2.8 mm. Preferably, a maximum force of about 30 N is required to deliver the intraocular lens through the cannula. More preferably, a maximum force of about 27 N, about 25 N, about 20 N or about 18 N is required to deliver the intraocular lens through the cannula.

Prior to deployment, at least a portion of the intraocular lens is coated with a viscoelastic composition according to any one of the embodiments, aspects, concepts, combinations or features of the present invention. The intraocular lens is loaded into the loadable chamber either before or after it is coated. The conduit is inserted through the passage. The actuator forces the intraocular lens through the passage and into the capsular bag. After the intraocular lens is deployed, the conduit is removed from the passage.

Typically, at least a portion of the viscoelastic composition is removed from the capsular bag and/or anterior chamber. A physiological solution is then used to fill the anterior chamber. The sclera and/or cornea are sutured to close the passage.

### EXAMPLES

### Example 1

### Preparation of Formulation 1

The following mixture was prepared and labeled as Formulation 1:
2.3% w/v hyaluronic acid (MW 1.98×10⁶)
0.8% w/v hydroxypropylmethylcellulose (MW 86,000)
4.4% w/v sorbitol
20 mM tris-[hydroxymethyl]aminomethane (“tris”)
purified water q. s. to 100 ml
pH 7.3
335 mOsm/Kg

### Example 2

### Preparation of Formulation 2

The following mixture was prepared and labeled as Formulation 2:
2% w/v hyaluronic acid (MW 1.98×10⁶)
0.8% w/v hydroxypropylmethylcellulose (MW 86,000)
4.4% w/v sorbitol
20 mM tris
purified water q. s. to 100 ml
pH 7.3
335 mOsm/Kg

### Example 3

### Preparation of Formulation 3

The following mixture was prepared and labeled as Formulation 3:
2% w/v hyaluronic acid (MW 1.98×10⁶)
1% w/v hydroxypropylmethylcellulose (MW 86,000)
4.4% w/v sorbitol
20 mM tris
purified water q. s. to 100 ml
pH 7.3
335 mOsm/Kg

### Example 4

### Preparation of Formulation 4

The following formulation was prepared and labeled as Formulation 4:
2.3% w/v hyaluronic acid (MW 1.98×10⁶)
0.8% w/v hydroxypropylmethylcellulose (MW 86,000)
4.4% w/v sorbitol
Purified water q. s. to 100 ml
<pH 7.3
<335 mOsm/Kg

### Example 5

### Preparation of Formulation 5

The following formulation was prepared and labeled as Formulation 5:
2.3% w/v hyaluronic acid (MW 1.98×10⁶)
0.8% w/v hydroxypropylmethylcellulose (MW 86,000)
Purified water q. s. to 100 ml
<pH7.3
<335 mOsm/Kg

### Example 6

### Preparation of Formulation 6

The following formulation was prepared and labeled as Formulation 6:
2.3% w/v hyaluronic acid (MW 1.98×10⁶)
0.8% w/v hydroxypropylmethylcellulose (MW 86,000)
20 mM tris
Purified water q. s. to 100 ml
pH 7.3
<335 mOsm/Kg

### Example 7

### Preparation of Formulation 7

The following formulation was prepared and labeled as Formulation 7:
2.3% w/v hyaluronic acid (MW 1.98×10⁶)
4.4% w/v sorbitol
20 mM tris
Purified water q. s. to 100 ml
pH 7.3
<335 mOsm/Kg

### Example 8

### Preparation of Formulation 8

A commercial sample of Viscoat® was labeled as Preparation 8.

### Example 9

### Free Radical Measurement of Formulations 1-8

The OH-scavenging activity of viscoelastic substances has been evaluated by the 2-deoxy-D-ribose (2-DR) oxidation method. This is a conventional method of OH-radical detection forming by the Fenton reaction, radiation or ultrasound. It is based on its reaction with 2-DR, which bring to the accumulation of 2-DR degradation products, especially malondialdehyde (MDA). The obtained product, after a thermo-activated reaction with thiobarbituric acid (TBA), produces a pink chromogen quantified by HPLC.

Stock solutions of 2-DR (40 mM), Fe²⁺/EDTA (10 mM), H₂O₂ (10 mM) in water (bubbled with N₂ for 30 min at room temperature) were prepared immediately before the experiment and stored on ice. An aliquot (500 μl) of the formulations 1 through 8 and water (used as control) was added to 900 μl phosphate buffer solution (0.1M, pH 7.4) and shaken by vortex until the solution was homogeneous. Then 200 μl of 2-DR, 200 μl of 1 mM Fe²⁺/EDTA and 200 μl H₂O₂ were added and the solution was shaken by vortex for 1 min. The sample solutions were incubated for 1 h at 37° C. and then added with 1 ml TBA (2% in 0.1M phosphate buffer ph 7.4) and lml TCA (2% in 0.1M phosphate buffer pH 7.4). The samples were again incubated at 100° C. for 30 min and cooled in ice. 100 μl of samples derived from Formulations 1 to 8, were diluted to 1 ml volume with mobile phase and injected onto HPLC.

The processed Formulations 1 to 8 were chromatographed over a C18 column to detect the pink chromogen product (TBA-MDA complex) using an UV-VIS detector at 532 nm. Chromatograms for Formulations 1 through 8 were compared to the chromatogram for the comparative standard. The percentage of production of TBA-MDA complex in Formulations 1 through 8 was compared to the standard solution (control), calculated and shown in Table 2. No production of TBA-MDA complex correspond to one hundred percent quenching of free radical activity. The amount of TBA-MDA complex in the comparative standard (control) represents zero percent because no quenching of the free radical activity occurred. Each of the formulations containing tris[hydroxymethyl]aminomethane and/or sorbitol had higher free radical quenching than samples without either. Tris[hydroxymethyl]aminomethane and sorbitol individually have free-radical quenching properties. The combination of Tris[hydroxymethyl]aminomethane and sorbitol have the best free-radical quenching properties.

TABLE 2
Percentage of Quenching of Free-Radical Activity

| | % w/v HA | % w/v | | | |
|---|---|---|---|---|---|
| | (mw | HPMC (mw | % w/v | Tris | % of |
| Formulations | 1.98 × 10⁶) | 8.6 × 10⁴) | Sorbitol | (mM) | quenching |
| 1 | 2.3 | 0.8 | 4.4 | 20 | 92 |
| 2 | 2 | 0.8 | 4.4 | 20 | 82 |
| 3 | 2 | 1.0 | 4.4 | 20 | 80 |
| 4 | 2.3 | 0.8 | 4.4 | — | 90 |
| 5 | 2.3 | 0.8 | — | — | 80 |
| 6 | 2.3 | 0.8 | — | 20 | 87.6 |
| 7 | 2.3 | — | 4.4 | 20 | 94 |
| 8 | — | — | — | — | 79 |
| Control | — | — | — | — | 0 |

Although preferred embodiments have been depicted and described in detail, it will be apparent to those skilled in the relevant art that the specification including the example are made without the intention of limiting the scope of the invention and that various, modifications, additions, substitutions, and the like can be made without departing from the spirit of the invention and these are therefore considered to be within the scope of the invention as defined in the claims which follow.

1. A viscoelastic composition comprising an aqueous solution having a minimum of about 0.01% w/v and a maximum of about 20% w/v of a viscoelastic polymer based upon the total volume of the viscoelastic composition and further having tris[hydroxymethyl]aminomethane.

2. The composition of claim 1, wherein the concentration of tris[hydroxymethyl]aminomethane is a maximum of about 50 mM and a minimum of about 0.1 mM based upon the total weight of the viscoelastic composition.

3. The composition of claim 1, further comprising a polyol.

4. The composition of claim 3, wherein at least one polyol is selected from the group comprising pentahydric alcohols, hexahydric alcohols and heptahydric alcohols and mixtures thereof.

5. The composition of claim 4, wherein the polyol is a hexahydric alcohol.

6. The composition of claim 5, wherein the polyol is mannitol.

7. The composition of claim 5, wherein the polyol is sorbitol.

8. The composition of claim 3, wherein the concentration of the polyol is a minimum of about 0.1% w/v and a maximum of about 15% w/v based upon the total volume of the viscoelastic composition.

9. The composition of claim 1, wherein the concentration of tris[hydroxymethyl]aminomethane is a minimum of about 0.5 mM and a maximum of about 30 mM.

10. The composition of claim 1, wherein the ratio of the viscosity of the viscoelastic composition to the viscosity of a comparable viscoelastic composition having no polyol and tris[hydroxymethyl]aminomethane is a minimum of about 1 and a maximum of about 2.5.

11. The composition of claim 1, wherein the percentage of quenching is a minimum of about 45% .

12. The composition of claim 1, wherein the viscoelastic polymer is selected from the group comprising hyaluronic acid, hydroxypropylmethylcellulose, polyacrilyc acid, carbopol, polyvinylalchol, polyvinylpirrolidone, condroitin sulfate, polycarbophil, methylcellulose, carboxymethylcellulose, hydroxyethylcellulose, hydroxypropylcellulose, ethylcellulose, polyethylene oxides, alginate, pectin, xanthan gum, dextrans, collagen and derivatives threof and salts thereof and combinations thereof.

13. The composition of claim 1, wherein the viscoelastic polymer comprises a polysaccharide.

14. The composition of claim 13, wherein the polysaccharide is selected from the group comprising hyaluronic acid, hydroxypropylmethylcellulose, condroitin sulfate, methylcellulose, carboxymethylcellulose, hydroxyethylcellulose, hydroxypropylcellulose, ethylcellulose, alginate, pectin, dextrans, collagen, proteoglycans, polyvinylpyrrolidone, keratin carrageenans and derivatives thereof and salts thereof and combinations thereof.

15. The composition of claim 1, wherein the viscoelastic polymer comprises alginate.

16. The composition of claim 15, wherein the concentration of alginate is a minimum of about 0.05% w/v and a maximum of about 9% w/v based upon the volume of the viscoelastic composition.

17. The composition of claim 15, wherein the average molecular weight of the alginate composition of yet minimum of about 50 kD and a maximum of about 5,000 kD.

18. The composition of claim 1, wherein the viscoelastic polymer comprises a mixture of hyaluronic acid and/or salts thereof and hydroxypropylmethylcellulose.

19. The composition of claim 18, wherein the concentration of hyaluronic acid and/or salts thereof is a minimum of about 0.1% w/v and a maximum of about 6% w/v based upon the volume of the viscoelastic composition.

20. The composition of claim 19, wherein the average molecular weight of the hyaluronic acid and/or salts thereof composition of yet minimum of about 500 kD and a maximum of about 5000 kD.

21. The composition of claim 18, wherein the concentration of hydroxypropylmethylcellulose is a minimum of about 0.05% w/v and a maximum of about 5% w/v based upon the volume of the viscoelastic composition.

22. The composition of claim 21, wherein the average molecular weight of the hydroxypropylmethylcellulose composition of yet minimum of about 10 kD and a maximum of about 120 kD.

23. The composition of claim 1, wherein the average molecular weight of the viscoelastic polymer is a minimum of about 20 kD and a maximum of about 5,000 kD.

24. The composition of claim 1, wherein the concentration of the viscoelastic polymer is a minimum amount of about 0.01% w/v and a maximum amount of about 20% w/v based upon the total weight of the viscoelastic composition.

25. The composition of claim 1, wherein the osmolality of the viscoelastic composition is a minimum of about 200 mOsmol/Kg and a maximum of about 400 mOsmol/Kg.

26. The composition of claim 1, wherein the zero-shear viscosity of the viscoelastic composition is a minimum of about 6·10⁴ cps and a maximum of about 4·10⁶ cps.

27. The composition of claim 1, wherein the high-shear viscosity of the viscoelastic composition is a minimum of about 500 cps and a maximum of about 2000 cps.

28. The composition of claim 1, wherein the pH of the viscoelastic composition is a minimum of about 5 and a maximum of about 8.

29. A method of temporarily maintaining space in a cavity in human tissue, the method comprising the steps of:
(a) injecting the viscoelastic composition of claim 1 into the cavity; and
(b) removing the viscoelastic composition from the cavity.

30. The process of claim 29, wherein the cavity is the anterior chamber of the eye or the capsular bag.

31. A method of protecting tissue from trauma during a surgical procedure, the method comprising the steps of:
(a) coating at least a portion of the tissue with the viscoelastic composition of claim 1;
(b) performing a surgical procedure near the tissue after the step of (a) coating; and
(c) removing at least a portion of the viscoelastic composition from the tissue after the step of (b) performing.

32. The method of claim 31, wherein the step of (a) coating covers at least a portion of the tissue in an anterior chamber of an eye.

33. The method of claim 31, wherein the step of (a) coating covers at least a portion of the tissue in a capsular bag of an eye.

34. A method of replacing a natural lens from an eye, the method comprising the steps of:
(a) providing a passage through a sclera into an anterior chamber of the eye;
(b) removing at least a portion of the aqueous humor from the anterior chamber;
(c) inserting the viscoelastic composition of claim 1 into the anterior chamber;
(d) phacoemulsifying a lens in the capsular bag of the eye;
(e) removing substantially all of the lens from the capsular bag;
(f) injecting the viscoelastic composition into the capsular bag; and
(g) inserting an intraocular lens into the capsular bag.

35. The method of claim 34, further comprising the step of (b) removing at least a portion of the viscoelastic composition from the capsular bag.

36. The method of claim 34, further comprising the step of (b) removing at least a portion of the viscoelastic composition from the anterior chamber.

37. The method of claim 36, further comprising the step of suturing the sclera after the step of (g) inserting an intraocular lens.

38. The method of claim 34, wherein the step of inserting comprises coating the intraocular lens with the viscoelastic composition and delivering the intraocular lens through a cannula.

39. The method of claim 38, wherein the cannula has a tip configured to be inserted into the capsular bag, wherein the tip of the cannula has an inner diameter that is a maximum of about 1 mm.

43. The method of claim 38, wherein the step of delivering the intraocular lens through the cannula requires a maximum force of about 30 N.

44. A package for a viscoelastic composition, the package comprising a syringe containing the viscoelastic composition of claim 1.

45. The package of claim 44, wherein the syringe has an outlet port, the package further comprises a cannula configured to sealably connect to the outlet port having a maximum inner diameter of about 2 mm.

46. The package of claim 44, wherein viscoelastic composition requires a maximum force of 30 N to pass through the cannula.
